(19) United States
(12) Patent Application Publication
(10) Pub. No.: US 2015/0166476 A1
(43) Pub. Date: Jun. 18, 2015

(54) METHODS FOR TREATING NEURODEGENERATIVE DISEASES ASSOCIATED WITH AGGREGATION OF AMYLOID-BETA

(71) Applicant: Yi-Cheng CHEN, Taipei (TW)

(72) Inventor: Yi-Cheng CHEN, Taipei (TW)

(21) Appl. No.: 14/109,948

(22) Filed: Dec. 17, 2013

(51) Int. Cl. C07C 323/22; A61K 31/192; A61K 45/06; A61K 31/122

(52) U.S. Cl. CPC C07C 323/22; A61K 31/122; A61K 31/192; A61K 45/06

(57) ABSTRACT

Disclosed herein is a method for prophylaxis or treatment of a neurodegenerative disease associated with aggregation of amyloid-beta (Aβ) in a subject. The method includes the step of, administering to the subject an effective amount of a compound having formula (I),

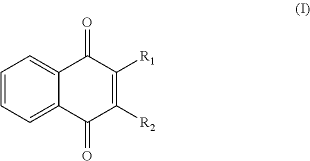

wherein, R₁ is selected from the group consisting of —SC₃H₆OH, —SC₂H₄COOH, —SCH₂CHOHCH₃, —SCH₂CHOHCH₂OH, —S(C₆H₄)OH, —SC₂H₄OH, —OH, and —NHC₂H₄(NC₂H₄OC₂H₄); and R₂ is H or CH₃.

### BACKGROUND OF RELATED ART

1. Technical Field

The present disclosure relates to novel use of menadione derivatives. Specifically, the present disclosure relates to the use of certain menadione derivatives for the treatment or prophylaxis of a neurodegenerative disease resulted from plaque formation.

2. Description of Related Art

Neurodegenerative diseases have become an important health issue in the modern society. According to the report of world health organization (WHO), more than 75% of elder population in the world will suffer some kinds of neurodegenerative disease in the year of 2025. Alzheimer's disease (AD) is the most common form of dementia and characterized by a progressive accumulation of intracellular and/or extracellular deposits of proteinaceous structures such as amyloid plaques in the brain of the affected patients. The appearance of amyloid plaques correlates with cognitive impairment such as memory loss, decrease in the ability to work out routine tasks, space and time disorientation, learning difficulties, reasoning disorientation, rapid mood changes and personality alteration, in the affected patients. Currently, there is no cure or treatment for such disease, and significant efforts have been made to identify compounds that may modulate the plaque formation.

Evidence suggests that amyloid plaques are resulted from aggregation of amyloid beta (Aβ) peptides, which gradually undergo a conformational conversion from either helix/or random coil into β-sheet in monomeric state, and consequently assemble into toxic Aβ aggregates or fibril. Hence, compound that stabilizes the conformation of monomeric Aβ or inhibit its misfolding or aggregation would be an ideal drug candidates for treating neurodegenerative diseases associated with plaque formation.

In view of the foregoing, there exist in the related art, a need for identifying compound(s) that may modulate the Aβ plaque formation, such compound(s) will be potential drug candidates for the manufacture of a medicament for the prophylaxis or treatment of neurodegenerative diseases associated with aggregation of Aβ.

### SUMMARY

This invention is based on the finding that certain menadione derivatives are capable of suppressing the aggregation of amyloid beta (Aβ), thereby preventing native form Aβ from aggregrating into amyloid plaque. Thus, these menadione derivatives are potential candidates as lead compounds for the manufacture of a medicament suitable for preventing or treating a neurodegenerative disease resulted from plaque formation.

Accordingly, the present disclosure aims to provide a method for the prophylaxis or treatment of a neurodegenerative disease associated with aggregation of amyloid-beta (Aβ) in a subject. The method includes the step of, administering to the subject a therapeutically effective amount of a compound of formula (I) or a pharmaceutically acceptable salt thereof, so as to inhibit or suppress the aggregation of Aβ,

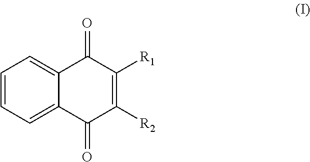

wherein,

R₁ is selected from the group consisting of —SC₃H₆OH, —SC₂H₄COOH, —SCH₂CHOHCH₃, —SCH₂CHOHCH₂OH, —S(C₆H₄)OH, —SC₂H₄OH, —OH, and —NHC₂H₄(NC₂H₄OC₂H₄); and R₂ is H or CH₃.

According to one preferred embodiment, R₁ is —SCH₂CHOHCH₂OH and R₂ is CH₃. In another preferred embodiment, R₁ is —SC₂H₄COOH and R₂ is CH₃. In still another preferred embodiment, R₁ is —S(C₆H₄)OH and R₂ is CH₃.

The neurodegenerative disease that may be treated by the method of the present disclosure is AD, vascular dementia, frontotemporal dementia, semantic dementia and dementia with Lewy bodies or Parkinson's disease (PD).

According to optional embodiments of the present disclosure, the method may further include the step of, administering to the subject an acetylcholinesterase inhibitor (AChEI), an Aβ inhibitor, or a muscarinic receptor agonist, either simultaneously or sequentially with the compound of formula (I) or a pharmaceutically acceptable salt thereof.

In some embodiments, the AChEI is any of alantamine, cymserine, donepezil, ER 127528, galantamine, ganstigmine, huperzine A, phenserine, phenethylnorcymserine, rivastigmine, RS 1259, SPH 1371, tacrine, thiacymserine, or zanapezil. In other embodiments, the Aβ inhibitor is any of bapineuzumab, PTB2, scyllo-inositol, PPI 1019, RS 0406, SP 233, EGCG, Exberyl-1, or SEN 606. The muscarinic receptor agonist is oxotremorine or xanomeline.

It is therefore the second aspect of this disclosure to provide a use of the compound of formal (I) as described above for manufacturing a medicament or a pharmaceutical composition for treating a neurodegenerative disease associated with aggregation of Aβ. The medicament or the pharmaceutical composition comprises an effective amount of the compound having the formula shown above; and a therapeutically acceptable excipient.

The compound of this invention is present at a level of about 0.1% to 99% by weight, based on the total weight of the pharmaceutical composition. In some embodiments, the compound of this invention is present at a level of at least 1% by weight, based on the total weight of the pharmaceutical composition. In certain embodiments, the compound of this invention is present at a level of at least 5% by weight, based on the total weight of the pharmaceutical composition. In still other embodiments, the compound of this invention is present at a level of at least 10% by weight, based on the total weight of the pharmaceutical composition. In still yet other embodiments, the compound of this invention is present at a level of at least 25% by weight, based on the total weight of the pharmaceutical composition.

In some embodiments, the medicament or the pharmaceutical composition of this invention further includes an agent that is known to improve the symptoms of a neurodegenerative disease associated with aggregation of Aβ. Examples of such agent include, but are not limited to, acetylcholinesterase inhibitor (AChEI), an Aβ inhibitor, or a muscarinic receptor agonist, and the like.

In some embodiments, the AChEI is any of alantamine, cymserine, donepezil, ER 127528, galantamine, ganstigmine, huperzine A, phenserine, phenethylnorcymserine, rivastigmine, RS 1259, SPH 1371, tacrine, thiacymserine, or zanapezil. In other embodiments, the Aβ inhibitor is any of bapineuzumab, PTB2, scyllo-inositol, PPI 1019, RS 0406, SP 233, EGCG, Exberyl-1, or SEN 606. The muscarinic receptor agonist is oxotremorine or xanomeline.

The details of one or more embodiments of the invention are set forth in the accompanying description below. Other features and advantages of the invention will be apparent from the detail descriptions, and from claims.

### BRIEF DESCRIPTION OF THE DRAWINGS

The present description will be better understood from the following detailed description read in light of the accompanying drawings, where:

FIG. 1 illustrating the ratio of Th-T fluorescence intensity (the aggregation level of Aβ1-40) of Aβ1-40 alone and VK3 compound/Aβ1-40 on day 5 in according to one embodiment of the present disclosure;

FIG. 2 illustrates the FT-IR spectra (the conformational states) for Aβ1-40 in the presence of the compound of formula (I) at day 1 and day 5 in accordance with one embodiment of the present disclosure;

FIG. 3 is a bar graph illustrating the effects of compound of formula (I) on Aβ1-40 induced cell death in accordance with one embodiment of the present disclosure; and

FIG. 4 is a bar graph illustrating the effects of compound of formula (I) on the levels of free radicals induced by Aβ1-40 (the main mechanism of cell death induced by Aβ aggregates) in accordance with one embodiment of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. DEFINITION

The term “an effective amount” as used herein refers to an amount effective, at dosages, and for periods of time necessary, to achieve the desired therapeutically result with respect to the treatment of a neurodegenerative disease associated with aggregation of Aβ.

The phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “administered”, “administering” or “administration” are used interchangeably herein to refer means either directly administering a bi-specific antibody or a composition of the present disclosure.

The term “subject” or “patient” refers to an animal including the human species that is treatable with the compositions and/or methods of the present disclosure. The term “subject” or “patient” intended to refer to both the male and female gender unless one gender is specifically indicated. Accordingly, the term “subject” or “patient” comprises any mammal which may benefit from treatment of cancer. Examples of a “subject” or “patient” include, but are not limited to, a human, rat, mouse, guinea pig, monkey, pig, goat, cow, horse, dog, cat, bird and fowl. In an exemplary embodiment, the patient is a human.

The singular forms “a”, “and”, and “the” are used herein to include plural referents unless the context clearly dictates otherwise.

### II. DESCRIPTION OF THE INVENTION

In one aspect, the present disclosure is directed to the use of certain menadione derivatives to inhibit the aggregation of a protein, particularly, amyloid-beta (Aβ), which aggregation is associated with a disease. In general, the protein aggregation process proceeds in a self-propagating manner, once initiated, an aggregation cascade ensues that involves induced conformation change and/or polymerization of further protein molecules, leading to the formation of toxic product that is resistant to proteolysis. The thus formed protein aggregation is thought to be the proximal cause of neurodegeneration diseases, such as AD, vascular dementia, frontotemporal dementia, semantic dementia, dementia with Lewy bodies and Parkinson's disease (PD).

The compounds of the present invention are derived from menadione, and may be synthesized in accordance with the method described by Chen et al (Bioorg. Med. Chem. Lett. (2002) 12: 2729-2732). Accordingly, 15 compounds were synthesized and tested for their capabilities in preventing Aβ from aggregating into toxic complex, and among them, 8 compounds were identified to possess anti-Aβ aggregation activities.

The compounds of the present invention have the following formula,

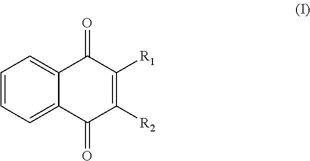

wherein,

R₁ is selected from the group consisting of —SC₃H₆OH, —SC₂H₄COOH, —SCH₂CHOHCH₃, —SCH₂CHOHCH₂OH, —S(C₆H₄)OH, —SC₂H₄OH, —OH, and —NHC₂H₄(NC₂H₄OC₂H₄); and R₂ is H or CH₃.

In one preferred embodiment, R₁ is —SCH₂CHOHCH₂OH and R₂ is CH₃. In another preferred embodiment, R₁ is —SC₂H₄COOH and R₂ is CH₃. In still another preferred embodiment, R₁ is —S(C₆H₄)OH and R₂ is CH₃.

Accordingly, the disclosure provides a pharmaceutical composition or a medicament for treating a neurodegenerative disease associated with the aggregation of Aβ. The neurodegenerative disease treatable by the pharmaceutical composition or the medicament of the present disclosure includes, but is not limited to, Alzheimer's disease (AD), vascular dementia, frontotemporal dementia, semantic dementia, dementia with Lewy bodies and Parkinson's disease (PD). The composition comprises an effective amount of the compound having formula (I) as shown above; and a pharmaceutically acceptable excipient.

Generally, the compound having formula (I) of this invention is present at a level of about 0.1% to 99% by weight, based on the total weight of the pharmaceutical composition. In some embodiments, the compound having formula (I) of this invention is present at a level of at least 1% by weight, based on the total weight of the pharmaceutical composition. In certain embodiments, the compound having formula (I) is present at a level of at least 5% by weight, based on the total weight of the pharmaceutical composition. In still other embodiments, the compound having formula (I) is present at a level of at least 10% by weight, based on the total weight of the pharmaceutical composition. In still yet other embodiments, the compound having formula (I) is present at a level of at least 25% by weight, based on the total weight of the pharmaceutical composition.

In some embodiments, the medicament of said pharmaceutical composition of this invention further includes an agent that is known to improve the symptoms of a neurodegenerative disease. Examples of such agent include, and are not limited to, AChEI, an Aβ inhibitor, or a muscarinic receptor agonist, and the like.

The medicament or said pharmaceutical composition is prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable excipients are those that are compatible with other ingredients in the formulation and biologically acceptable.

The compounds of this invention (e.g., the compound having formula (I) as shown above) may be administered orally, parenterally, transdermally, rectally or by inhalation, alone or in combination with conventional pharmaceutically acceptable excipients. In preferred embodiments, the compounds of this invention are administered parenterally to the subject.

Applicable solid excipients may include one or more substances that may also act as flavoring agents, lubricants, solubilizers, suspending agents, fillers, glidants, compression aids, binders or tablet-disintegrating agents or an encapsulating material. In powders, the excipient is a finely divided solid that is in admixture with the finely divided active ingredient. In tablets, the active ingredient is mixed with an excipient having the necessary compression properties in suitable proportions and compacted in the shape and size desired. The powders and tablets preferably contain up to 99% of the active ingredient. Suitable solid excipient includes, for example, calcium phosphate, magnesium stearate, talc, sugars, lactose, dextrin, starch, gelatin, cellulose, methyl cellulose, sodium carboxymethyl cellulose, polyvinylpyrrolidine and the like.

The compounds of the present invention may also be formulated into liquid pharmaceutical compositions, which are sterile solutions, or suspensions that can be administered by, for example, intravenous, intramuscular, subcutaneous, intraperitoneal or intra-cerebella injection. Oral administration may be either liquid or solid composition form.

The medicament or said pharmaceutical compositions of this invention may be formulated into a variety of dosage forms for topical application. A wide variety of dermatologically acceptable inert excipients well known to the art may be employed. The topical compositions may include liquids, creams, lotions, ointments, gels, sprays, aerosols, skin patches, and the like. Typical inert excipients may be, for example, water, ethyl alcohol, polyvinyl pyrrolidone, propylene glycol, mineral oil, stearyl alcohol and gel-producing substances. All of the above dosages forms and excipients are well known to the pharmaceutical art. The choice of the dosage form is not critical to the efficacy of the composition described herein.

The medicament or said pharmaceutical compositions of this invention may also be formulated in a variety of dosage forms for mucosal application, such as buccal and/or sublingual drug dosage units for drug delivery through oral mucosal membranes. A wide variety of biodegradable polymeric excipients may be used that are pharmaceutically acceptable, provide both a suitable degree of adhesion and the desired drug release profile, and are compatible with the active agents to be administered and any other components that may be present in the buccal and/or sublingual drug dosage units. Generally, the polymeric excipient comprises hydrophilic polymers that adhere to the wet surface of the oral mucosa. Examples of polymeric excipients include, but are not limited to, acrylic acid polymers and copolymers; hydrolyzed polyvinylalcohol; polyethylene oxides; polyacrylates; vinyl polymers and copolymers; polyvinylpyrrolidone; dextran; guar gum; pectins; starches; and cellulosic polymers.

Accordingly, this invention also provides methods of treating mammals, preferably humans, of a neurodegenerative disease associated with aggregation of Aβ, which comprises the administration of the medicament or said pharmaceutical composition of this invention that contains a compound having formula (I) as shown above. Such medicament or composition is administered to a mammal, preferably human, by any route that may effectively transports the active ingredient(s) of the composition to the appropriate or desired site of action, such as oral, nasal, pulmonary, transdermal, such as passive or iontophoretic delivery, or parenteral, e.g., rectal, depot, subcutaneous, intravenous, intramuscular, intranasal, intra-cerebella, ophthalmic solution or an ointment. Further, the administration of the compound of this invention with other active ingredients may be concurrent or simultaneous.

In some embodiments, the effective dose administered to the subject is from about 1 to 100 mg/Kg body weight of the subject, such as about 10, 20, 30, 40, 50, 60, 70, 80, 90 or 100 mg/Kg body weight of the subject, preferably about 50 to 70 mg/Kg body weight of the subject, such as 50, 60 or 70 mg/Kg body weight of the subject; most preferably about 50 mg/Kg body weight of the subject. The dose can be administered in a single aliquot, or alternatively in more than one aliquot.

According to optional embodiments of the present disclosure, the method may further include the step of, administering to the subject an acetylcholinesterase inhibitor (AChEI), an Aβ inhibitor, or a muscarinic receptor agonist, either simultaneously or sequentially with the compound of formula (I) as described above or a pharmaceutically acceptable salt thereof.

In some embodiments, the AChEI is any of alantamine, cymserine, donepezil, ER 127528, galantamine, ganstigmine, huperzine A, phenserine, phenethylnorcymserine, rivastigmine, RS 1259, SPH 1371, tacrine, thiacymserine, or zanapezil. In other embodiments, the Aβ inhibitor is any of bapineuzumab, PTB2, scyllo-inositol, PPI 1019, RS 0406, SP 233, EGCG, Exberyl-1, or SEN 606. The muscarinic receptor agonist is oxotremorine or xanomeline.

The present invention will now be described more specifically with reference to the following embodiments, which are provided for the purpose of demonstration rather than limitation.

### EXAMPLES

### Materials and Methods

Synthesis of the Compounds of Formula (I)

The compounds of formula (I) were synthesized in accordance with the method described by Chen et al (Bioorg. Med. Chem. Lett. (2002) 12: 2729-2732), and are summarized as bellow.

| | |
|---|---|
| | (I) |
| 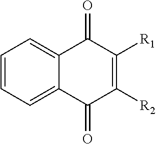 | |

| Name | R₁ | R₂ |
|---|---|---|
| VK3-1 | —SC₂H₄OH | —CH₃ |
| VK3-2 | —SC₃H₆OH | —CH₃ |
| VK3-3 | —SC₄H₈OH | —CH₃ |
| VK3-4 | —SC₆H₁₂OH | —CH₃ |
| VK3-5 | —SC₁₁H₂₂OH | —CH₃ |
| VK3-6 | —SC₂H₄COOH | —CH₃ |
| VK3-8 | —SCH₂CHOHCH₃ | —CH₃ |
| VK3-9 | —SCH₂CHOHCH₂OH | —CH₃ |
| VK3-10 | —S(C₆H₄)OH | —CH₃ |
| VK3-199 | —SC₂H₄OH | —H |
| VK3-221 | —OH | —CH₃ |
| VK3-231 | —SC₂H₄OH | —SC₂H₄OH |
| VK3-232 | —SCH₂CHOHCH₂OH | —SCH₂CHOHCH₂OH |
| VK3-233-2d | —SC₆H₁₂OH | —SC₆H₁₂OH |
| VK3-224 | —NHC₂H₄(NC₂H₄OC₂H₄) | —CH₃ |

Synthesis and Purification of Aβ1-40

Aβ1-40 peptide was synthesized in a solid-phase synthesizer (ABI 433A) using standard FMOC protocols with HMP resin. After cleavage from the resin with a mixture of trifluoroacetic acid/H₂O/ethanedithol, thioanisole/phenol, the peptides were extracted with 1:1 (v:v) ether: H₂O containing 0.1% 2-mercapthanol. The synthesized Aβ1-40 peptides were purified using a C₁₈ reverse-phase column with a linear gradient from 0% to 78% acetonitrile. Peptide purity was over 95% as identified by matrix-assisted laser desorption/ionization-time of flight mass spectrometry. 1 mg of Aβ1-40 peptide was dissolved in 1 mL trifluoroethanol, and centrifuged at a speed of 20,000×g to remove any insoluble particles. The thus obtained Aβ1-40 solution was then dried under nitrogen gas and re-suspended in 1 mL phosphate buffer (pH 7.4) to provide a stock solution, and was stored at −80° C. until used.

Free Radical Assay

The level of free radicals (H₂O₂) induced by Aβ1-40 peptide in cell free conditions was analyzed using the dichlorofluorescein diacetate (DCFH-DA) assay. DCF-DA was deacetylated with 50% (v/v) 0.05 M NaOH for 30 min and neutralized (pH 7.5) to a final concentration of 200 μM as a stock solution. This stock solution was kept on ice in the dark until future use. The reactions were carried out in a 96-well plate (200 μL/well) containing 25 μM of Aβ1-40 diluted from the stock solution, 20 μM deacetylated DCF, 5 μM horseradish peroxidase, in Dulbecco's phosphate-buffered saline, pH 7.5. To determine the inhibitory effects of the compound of formula (I) on free radical formation, various concentrations of the compound of formula (I) were added and incubated at 37° C. Fluoresence readings were recorded on a microplate reader (FlexSTation 3, MD) with the excitation wavelength of 485 nm and the emission wavelength of 530 nm. The fluorescence intensity of DCF (H₂O₂ level) was measured every 6 hr and from 0 to 72 hr.

Peptide Aggregation Assay

Thioflavin-T (ThT) was used to monitor the aggregation state of Aβ1-40. 25 μM of Aβ1-40 was freshly diluted from the peptide stock solution in phosphate buffer (pH 7.4), for peptide aggregation assay. All samples containing a peptide concentration of 25 μM in the absence or presence of 100 ng/mL the compound of formula (I) and 3 μM ThT were incubated at 37° C. Samples containing either Aβ peptide only (control), or Aβ with the compound of formula (I), taking daily from day 0 to day 7, were used to measure the ThT sensitivity. The fluorescence measurement was performed on a microplate reader (FlexSTation 3, MD), with excitation and emission wavelength respectively set at 440 nm and 485 nm.

Cell Culture

Human blastoma SH-SY5Y cells were cultured in minimum essential medium supplemented with 10% (v/v) heat-inactivated fetal bovine serum, 50% (v/v) F-12 nutrient mixture, and 1% (v/v) antibiotic mixture comprised of penicillin and streptomycin. Cells were kept at 37° C. in a humidified atmosphere of 5% CO₂. SH-SY5Y cells were plated at a density of 1×10⁵ viable cells per well in 96-well plates for future analysis.

Cell Viability Assay

The cell viability was determined by the WST-1 assay. 500 μM of Aβ1-40 peptide stock solution were prepared by dissolving 1 mg Aβ1-40 in 1 mL trifluoroethanol and centrifuging to remove any insoluble particles. The peptide solution was then dried under nitrogen gas and re-dissolved in dimethyl sulfoxide, and incubated at 4° C. for 12 hr to provide the final peptide stock solution. For the viability assay, cells (1×10⁵ cells/well in a 96-well microtiter plate) were treated with either 25 μM Aβ peptides only (as a positive control), or with the combination of 25 μM Aβ peptides and the compound of formula (I), in a concentration ranged from 1 to 1,000 ng/mL. The reaction was performed in a total volume of 200 μL per well for 24 hr at 37° C. in a humidified atmosphere containing 5% CO₂ before cell viability was assayed. The WST-1 solution (10 μL) was added to each well, and the wells were incubated for another 4-5 hr at room temperature. The optical density was measured at 405 nm using a microplate reader.

Fourier-Transform Infrared (FT-IR) Spectroscopy

The secondary structure of Aβ1-40 with or without the compound of formula (I) was investigated using FT-IR spectrometer (Jasco, FT-IR/4100) equipped with an attenuated reflection accessory to determine the conformation of Aβ1-40 during the aggregative process. 100 μL of 0.1 mM Aβ solution was coated on ZnSe crystals and dried overnight in a desiccators at room temperature. The spectra were recorded at 1,500 to 1,800 cm⁻¹ with a 1 cm⁻¹ interval. The peak was identified from the first derivation of the IR spectrum in the amide I region, and the secondary structure was analyzed using Original 6.0 software.

### Example 1

### The Compound of Formula (I) Inhibits the Aggregation of Aβ1-40

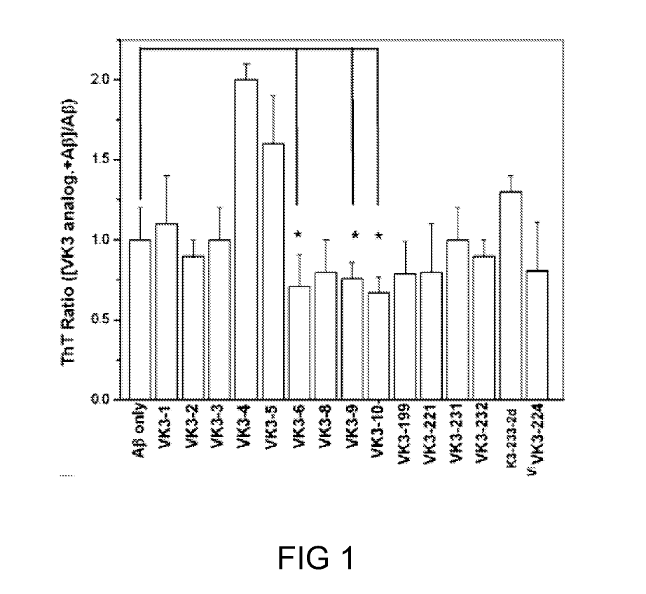

In this example, the effects of the compound of formula (I) on the aggregation of Aβ1-40 were investigated using the Th-T fluorescence assay described above in the Material and Methods section. Results are depicted in FIG. 1.

As evident from FIG. 1, several compounds of formula (I) of the present disclosure, including VK3-2, VK3-6, VK3-8, VK3-9, VK3-10, VK3-199, VK3-221, and VK3-224 compounds, were all capable of preventing Aβ1-40 from aggregation. Among them, VK3-6, VK3-9, and VK3-10 were most potent. By contrast, some compounds of formula (I) of the present disclosure, including VK3-1, VK3-4, VK3-5 and VK3-233-2d, were capable of enhancing the aggregation of Aβ1-40.

### Example 2

### Effects of Compound of Formula (I) on the Secondary Structure of Aβ1-40

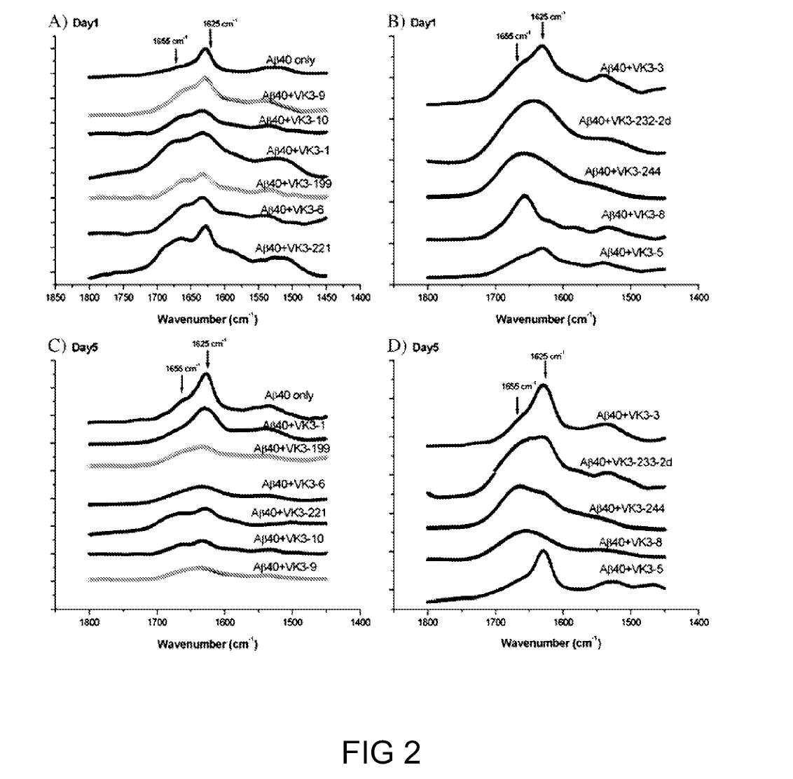

It is known that during the aggregation process, the conformation of Aβ1-40 is converted to either helix or random coil β-sheet. Hence, in this example, the effects of compounds of formula (I) on the conformation of Aβ1-40 were investigated. Results are depicted in FIG. 2.

The conformation of Aβ1-40 in the presence of VK3-2, VK3-6, VK3-8, VK3-9, VK3-10, VK3-199, VK3-221, and VK3-224 compounds remained mostly in random coil conformation on both days 1 and 5, as the 1650 cm⁻¹ major peak was an indication of random coil. By contrast, the 1650 cm⁻¹ peak that appeared in the FT-IR spectra of Aβ1-40 alone or in the presence of VK3-1, VK3-3, VK3-5, and VK3-232-2d on day 1 shifted to 1625 cm⁻¹ on day 5, which indicated that Aβ1-40 had converted to β-sheet structure. The results are consistent with the finding in Example 1, in which VK3-6, VK3-9, VK3-10, VK3-199, and VK3-221 compounds inhibited the conformation change of Aβ1-40.

### Example 3

### Effects of Compound of Formula (I) on Aβ1-40 Induced Cell Death

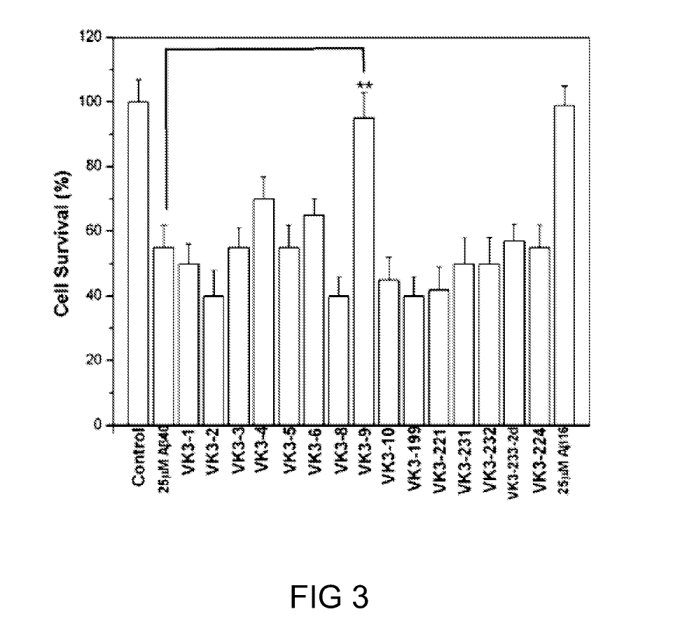

In this example, the effects of compounds of formula (I) on Aβ1-40 induced cell toxicity were investigated by cell viability assay. Results are depicted in FIG. 3.

Unlike the findings from aggregation and secondary structure studies as described above in FIGS. 1 and 2, cell viability assay as illustrated in FIG. 3 indicated that only compound VK3-9 exhibited the ability of preventing Aβ1-40 induced cell death at concentration below 100 ng/mL, whereas the rest of compound of formula (I) including VK3-1, VK3-2, VK3-3, VK3-4, VK3-5, VK3-6, VK3-8, VK3-10, VK3-119, VK3-221 and VK3-231, had smaller effects. As to compounds VK3-232, VK3-233-2d, and VK3-224, their effects as measured by cell viability assay showed no difference from that of the control (i.e., 25 μM Aβ1-40 alone). Further studies indicated that compound VK3-9 suppressed Aβ1-40 induced cell death in a dose-dependent manner, cell survival rate increased to 50% with 1 ng/mL VK3-9, 88% with 100 ng/mL VK3-9, and to 92% with 1,000 ng/mL VK3-9.

### Example 4

### Compound of Formula (I) Attenuates Aβ1-40 Induced Free Radical Formation

As Aβ induced free radicals had been proposed to be one of the possible mechanisms for causing cell death, hence the role of compound of formula (I) on Aβ1-40 induced free radical generation was investigated using the dichlorofluorescein diacetate (DCF-DA) assay.

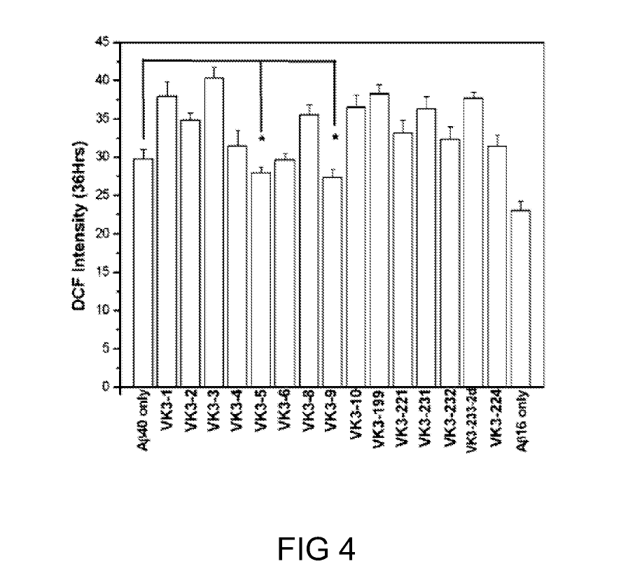

As depicted in FIG. 4, DCF fluorescence intensity of Aβ1-40 was reduced in the presence of compound VK3-5 or VK3-9; which indicates that the Aβ1-40 induced free radical levels were effectively inhibited by either compound VK3-5 or VK3-9. On the other hand, VK3-1, VK3-2, VK3-3, VK3-8, VK3-10, VK3-119, VK3-231 and VK3-232-2d, resulted even more free radicals as compared with the control (i.e., Aβ1-40 alone). Results from this example also provide a possible explanation that while VK3-10 was capable of preventing Aβ from aggregation, yet it failed to protect cells from Aβ induced cell death.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for the prophylaxis or treatment of a neurodegenerative disease associated with aggregation of amyloid-beta (Aβ) in a subject comprising administering to the subject a an effective amount of a compound of formula (I) or a pharmaceutically acceptable salt thereof, 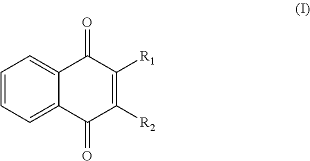
wherein,
R₁ is selected from the group consisting of —SC₃H₆OH, —SC₂H₄COOH, —SCH₂CHOHCH₃, —SCH₂CHOHCH₂OH, —S(C₆H₄)OH, —SC₂H₄OH, —OH, and —NHC₂H₄(NC₂H₄OC₂H₄); and R₂ is H or CH₃.

2. The method of claim 1, wherein R₁ is —SCH₂CHOHCH₂OH and R₂ is CH₃.

3. The method of claim 1, wherein R₁ is —SC₂H₄COOH and R₂ is CH₃.

4. The method of claim 1, wherein R₁ is —S(C₆H₄)OH and R₂ is CH₃.

5. The method of claim 1, wherein the neurodegenerative disease is Alzheimer's disease, vascular dementia, frontotemporal dementia, semantic dementia and dementia with Lewy bodies or Parkinson's disease.

7. The method of claim 1, further comprising administering to the subject an acetylcholinesterase inhibitor (AChEI), an Aβ inhibitor, or a muscarinic receptor agonist.

8. The method of claim 7, wherein the AChEI is any of alantamine, cymserine, donepezil, ER 127528, galantamine, ganstigmine, huperzine A, phenserine, phenethylnorcymserine, rivastigmine, RS 1259, SPH 1371, tacrine, thiacymserine, or zanapezil.

9. The method of claim 7, wherein the Aβ inhibitor is any of bapineuzumab, PTB2, scyllo-inositol, PPI 1019, RS 0406, SP 233, EGCG, Exberyl-1, or SEN 606.

10. The method of claim 7, wherein the muscarinic receptor agonist is oxotremorine or xanomeline.
